(19) United States
(12) Patent Application Publication
(10) Pub. No.: US 2025/0255545 A1
(43) Pub. Date: Aug. 14, 2025

(54) SYSTEM AND METHOD FOR LONGITUDINAL APNEA MONITORING USING SPO2 SIGNALS

(71) Applicant: UNIVERSITY OF SOUTH FLORIDA, Tampa, FL (US)

(72) Inventors: Trung Le, Tampa, FL (US); Quoc Nguyen, Tampa, FL (US)

(21) Appl. No.: 19/049,974

(22) Filed: Feb. 10, 2025

Related U.S. Application Data
(60) Provisional application No. 63/551,782, filed on Feb. 9, 2024.

(51) Int. Cl. A61B 5/00; A61B 5/024; A61B 5/1455; G16H 40/63; G16H 50/30

(52) U.S. Cl. CPC A61B 5/4818; A61B 5/02416; A61B 5/14551; A61B 5/4812; A61B 5/7253; G16H 40/63; G16H 50/30; A61B 2560/045

(57) ABSTRACT

A system and method for monitoring sleep apnea in cancer patients undergoing treatment are disclosed. The system includes a device to collect SpO2 signals from a patient over multiple sleep sessions, a gateway to receive and process the SpO2 signals to generate formatted SpO2 data, an apnea monitoring service to determine an apnea measure based on the formatted SpO2 data, and a user service to provide a longitudinal progression of the apnea measure. The method involves collecting SpO2 signals, processing them at a gateway, determining an apnea measure, and providing a longitudinal progression of the apnea measure over multiple sleep sessions. The system and method enable efficient monitoring and analysis of sleep apnea in cancer patients during treatment.

### CROSS-REFERENCE TO RELATED APPLICATIONS

This application claims priority to U.S. Application No. 63/551,782, filed Feb. 9, 2024, which is hereby incorporated by reference in its entirety.

### BACKGROUND

Cancer patients undergoing treatment often face various health challenges, including sleep-disordered breathing. Monitoring these patients' physiological parameters, particularly during sleep, can provide valuable insights for healthcare professionals. However, traditional in-lab sleep diagnostic methods, such as polysomnography (PSG), present limitations in terms of real-time monitoring capabilities and patient comfort.

There is a growing interest in developing alternative monitoring systems that can be used at home or remotely. Such systems could potentially improve accessibility, reduce costs, and facilitate long-term monitoring and collection of longitudinal data. This is particularly relevant in the context of cancer treatment, where continuous monitoring may be beneficial for effective management and follow-up care.

Sleep monitoring plays a role in cancer treatment monitoring, as sleep disorders can impact patient outcomes and quality of life during treatment. Sleep-disordered breathing is linked to cancer outcomes during treatment, with implications for patient care and prognosis. Research indicates that sleep disorders, particularly obstructive sleep apnea (OSA), are prevalent among cancer patients and can impact treatment efficacy and overall patient well-being.

The relationship between sleep-disordered breathing and cancer outcomes is multifaceted. Sleep disturbances can exacerbate cancer-related fatigue, which is one of the common and distressing symptoms reported by cancer patients undergoing treatment. This fatigue can lead to reduced physical activity, decreased quality of life, and potentially lower adherence to treatment regimens.

Sleep-disordered breathing, especially OSA, is associated with intermittent hypoxia and sleep fragmentation. These conditions can trigger systemic inflammation and oxidative stress, which may promote tumor growth and metastasis. Some studies suggest that the hypoxic environment created by sleep apnea events can stimulate angiogenesis and alter gene expression in ways that favor tumor progression.

The impact of sleep-disordered breathing on the immune system is another factor in cancer outcomes. Quality sleep is beneficial for maintaining a robust immune response, which is useful for fighting cancer and recovering from treatment side effects. Sleep disturbances can compromise immune function, potentially reducing the body's ability to combat cancer cells and increasing susceptibility to infections during treatment.

Additionally, sleep-disordered breathing may affect the pharmacokinetics of cancer treatments. Altered sleep patterns and associated physiological changes can influence drug metabolism and efficacy, potentially impacting the effectiveness of chemotherapy and other cancer treatments.

The presence of sleep-disordered breathing in cancer patients is also associated with increased cardiovascular risk. This is particularly significant as many cancer treatments, such as certain chemotherapies and radiation therapies, can have cardiotoxic effects. The combination of sleep disorders and cancer treatments may compound cardiovascular risks, potentially leading to complications that can interrupt or alter the course of cancer treatment.

Moreover, the psychological impact of sleep disorders should not be overlooked. Poor sleep quality and quantity can contribute to mood disorders such as anxiety and depression, which are already prevalent in cancer patients. These psychological factors can influence treatment decisions, coping mechanisms, and overall outcomes.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

Aspects of the described technology may provide a system comprising a device to collect SpO2 signals from a cancer patient undergoing treatment over multiple sleep sessions, a gateway to connect to the device via a local connection to receive and process the SpO2 signals, an apnea monitoring service to determine an apnea measure based on the formatted SpO2 data, and a user service to provide a longitudinal progression of the apnea measure. Further aspects may provide a method for collecting SpO2 signals from a cancer patient over multiple sleep sessions, processing and transmitting the signals via a gateway, determining an apnea measure based on the formatted data and providing a longitudinal progression of the apnea measure. Additional aspects may provide a non-transitory computer-readable medium storing instructions to perform operations including collecting SpO2 signals from a cancer patient over multiple sleep sessions, processing and transmitting the signals via a gateway, determining an apnea measure, and providing a longitudinal progression of the apnea measure.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1 illustrates a flowchart of a method for monitoring SpO2 signals from cancer patients, according to aspects of the present disclosure.

FIG. 2 illustrates a system architecture comprising three main tiers for processing and analyzing SpO2 data, in accordance with some embodiments described herein.

FIG. 3A and FIG. 3B illustrate orthogonal views of a sensor device for collecting SpO2 data, according to an embodiment of the present disclosure.

FIG. 4 illustrates a flowchart of a method for processing and transmitting SpO2 data, in accordance with some embodiments described herein.

FIG. 5 shows a bounce diagram illustrating data flow and interactions in a sleep monitoring system, according to aspects of the present disclosure.

FIG. 6 depicts a cross-correlation graph comparing SpO2 data collected from two different systems, in accordance with some embodiments described herein.

FIG. 7 illustrates a monitoring dashboard showing real-time physiological signal data, according to an embodiment of the present disclosure.

FIG. 8 illustrates a sleep monitoring system with multiple interconnected components, in accordance with some embodiments described herein.

FIG. 9 illustrates a system architecture for monitoring and analyzing physiological data, according to aspects of the present disclosure.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

There is a growing recognition of the need for alternative monitoring systems that can be used at home or remotely. Such systems could potentially improve accessibility, reduce costs, and facilitate long-term monitoring and the collection of longitudinal data. This is particularly relevant in the context of cancer treatment, where continuous monitoring is beneficial for effective management and follow-up care.

Additionally, the integration of advanced data analytics and machine learning algorithms in healthcare monitoring systems has shown promise in providing valuable insights and predictive capabilities. However, the implementation of such technologies in real-time, continuous monitoring systems for cancer patients presents challenges in terms of data processing, storage, and interpretation.

As the healthcare industry continues to evolve, there is an ongoing effort to develop more patient-centric approaches to monitoring and treatment. This includes the design of devices and systems that prioritize ease of use, comfort, and non-intrusiveness, while still providing accurate and comprehensive data for healthcare professionals.

In light of these connections, monitoring and managing sleep-disordered breathing in cancer patients may play a crucial role in improving treatment outcomes. Early detection and intervention for sleep disorders could potentially enhance treatment efficacy, reduce complications, and improve quality of life for cancer patients undergoing treatment. This underscores the importance of integrating sleep assessment and management into comprehensive cancer care protocols.

While the described technology is particularly beneficial for monitoring sleep patterns in cancer patients undergoing treatment, it may be applied to a wide range of sleep monitoring scenarios. The use of cancer patient monitoring serves as an illustrative example to demonstrate the capabilities and potential benefits of the system. However, the underlying principles and methodologies can be adapted and utilized in various other contexts where sleep monitoring is valuable. For instance, the system may be employed in monitoring sleep patterns of individuals with chronic respiratory conditions such as chronic obstructive pulmonary disease (COPD) or asthma, where nighttime breathing difficulties can significantly impact sleep quality and overall health. Another potential application may be in the field of sports medicine, where athletes' sleep patterns and quality can be monitored to optimize performance and recovery. The system could also be valuable in geriatric care settings, helping to identify and manage sleep disorders in elderly individuals, which can be indicative of or contribute to cognitive decline and other health issues. In each of these scenarios, the ability to collect longitudinal SpO2 data and provide comprehensive sleep analysis can offer valuable insights for healthcare providers, potentially leading to more personalized and effective treatment strategies.

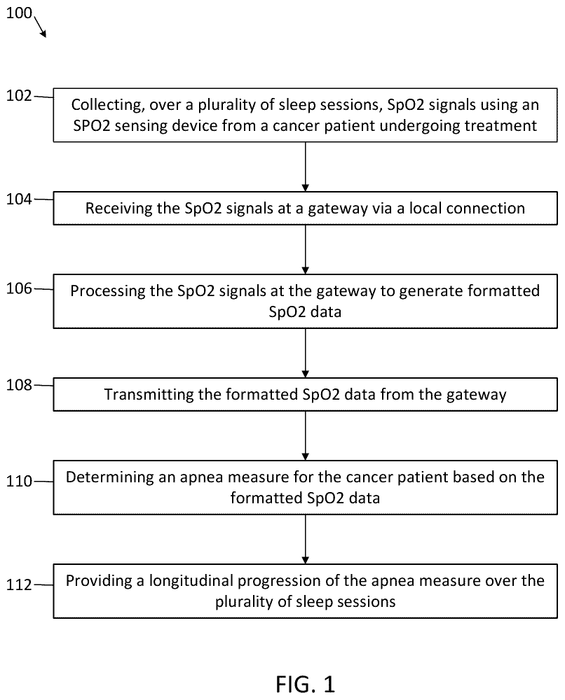

FIG. 1 illustrates a flowchart of the method 100, which comprises several steps for collecting, processing, and analyzing SpO2 data to provide valuable insights into a patient's sleep patterns and potential apnea conditions.

The method 100 begins at a block 102, which involves collecting SpO2 signals over multiple sleep sessions from a cancer patient undergoing treatment using an SpO2 sensing device. This device may include a pulse oximeter with an SpO2 sensor that gathers analog sensor data. A pulse oximeter may operate by emitting light at two different wavelengths, typically red and infrared, through a translucent part of the patient's body, such as a fingertip or earlobe. As the light passes through the body tissue, some of it is absorbed by blood and soft tissues, depending on the concentration of oxygenated and deoxygenated hemoglobin. The device may then measure the amount of light that is transmitted through the tissue and reaches the detector on the other side. The ratio of light absorption at these two wavelengths may correlate to the proportion of oxygenated hemoglobin in the arterial blood. Pulse oximeters may take advantage of the pulsatile nature of arterial blood flow to distinguish between absorption caused by arterial blood and that caused by other tissues. By analyzing the time-varying component of the absorption signal, the device may isolate the absorption specifically due to arterial blood.

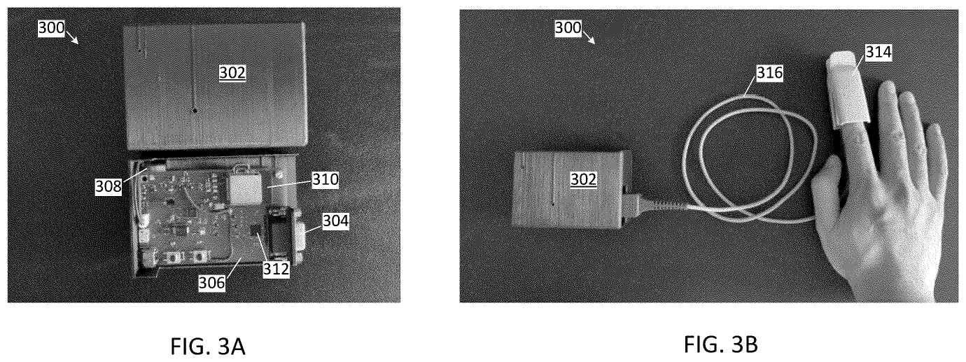

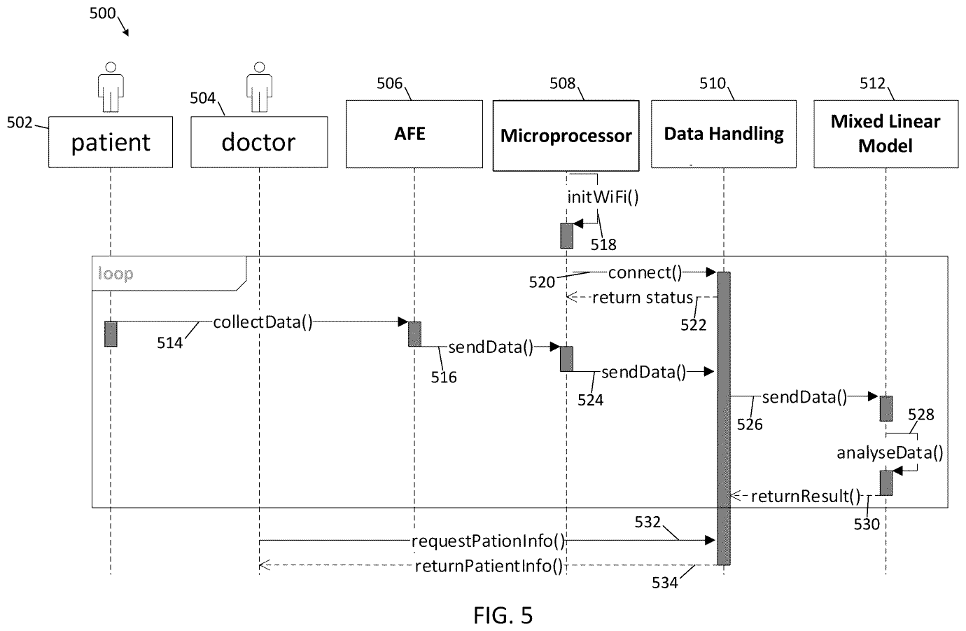

The digital sensor data is provided to a microcontroller (which may be implemented as described with respect to microcontroller 310 in FIG. 3 or microcontroller 508 in FIG. 5). The microcontroller can format the data according to a communication protocol. The microcontroller in the pulse oximeter may use this information to calculate the ratio of oxygenated to deoxygenated hemoglobin in the arterial blood. This ratio may then be converted into a SpO2 percentage, which represents the oxygen saturation of the blood. In some cases, the microcontroller may also provide additional information, such as heart rate, by analyzing the frequency of the pulsatile signal. Some examples may perform operations to reduce motion artifacts and improve accuracy in low perfusion states. In some implementations, the microcontroller may transmit the raw or partially processed SpO2 data to a server for further analysis and processing.

The microcontroller can format the data according to a communication protocol. The microcontroller in the pulse oximeter may use this information to calculate the ratio of oxygenated to deoxygenated hemoglobin in the arterial blood. This ratio may then be converted into a SpO2 percentage, which represents the oxygen saturation of the blood. In some cases, the microcontroller may also provide additional information, such as heart rate, by analyzing the frequency of the pulsatile signal. Some examples may perform operations to reduce motion artifacts and improve accuracy in low perfusion states. In some implementations, the microcontroller may transmit the raw or partially processed SpO2 data to a server for further analysis and processing.

In some cases, the microcontroller may transmit the processed SpO2 signals to a gateway via a wireless communication protocol. In some implementations, the microcontroller may utilize various wireless communication protocols to transmit the processed SpO2 signals to the gateway. These protocols may include Bluetooth Low Energy (BLE), Wi-Fi, Zigbee, or LoRaWAN. Bluetooth Low Energy may be suitable for short-range communication, providing low power consumption and compatibility with many mobile devices. Wi-Fi may offer higher data transfer rates and longer range compared to BLE. Zigbee may provide a mesh network capability, allowing multiple devices to communicate with each other and extend the range of the network. LoRaWAN may offer long-range communication with low power consumption. In some implementations, the microcontroller may be capable of supporting multiple wireless protocols, allowing for flexibility in different monitoring scenarios. The choice of protocol may depend on factors such as power consumption requirements, data transmission range, data rate needs, and integration with existing systems.

In some implementations, the system may utilize different wireless communication protocols depending on the operational mode of the SpO2 sensing device. For instance, Bluetooth Low Energy (BLE) may be employed in batch mode, while Wi-Fi may be used in streaming mode. When operating in batch mode, the system may leverage BLE's low power consumption characteristics. In this mode, the SpO2 sensing device may accumulate data over a predetermined period, such as several hours or overnight, before transmitting it to the gateway. BLE's energy efficiency may allow for extended battery life of the sensing device, which may be particularly beneficial for long-term monitoring scenarios. The lower data rate of BLE may be sufficient for transmitting the accumulated batch data periodically. In streaming mode, the system may switch to Wi-Fi communication. Wi-Fi's higher bandwidth capabilities may be advantageous for transmitting continuous, real-time SpO2 data streams. This mode may allow healthcare providers to monitor patients' SpO2 levels in real-time, which may be useful for immediate intervention if abnormalities are detected. The higher power consumption of Wi-Fi may be less of a concern in streaming mode, as the device may be expected to operate for shorter durations or could be connected to a power source.

The system may include logic to automatically switch between these protocols based on the current operational mode. For example, when a healthcare provider initiates a real-time monitoring session, the device may switch from BLE to Wi-Fi to facilitate streaming. Conversely, when the streaming session ends, the device may revert to BLE for energy-efficient batch data collection. This dual-protocol approach may offer flexibility in data transmission, balancing power efficiency with real-time monitoring capabilities. It may allow the system to adapt to different monitoring scenarios, from long-term sleep studies to immediate patient assessment, enhancing its utility in various clinical contexts.

At a block 104, the method 100 continues with the sensing device transmitting data to a gateway and receiving the SpO2 signals at the gateway through the local connection (e.g., BLE, Wi-Fi, Zigbee, Z-Wave, etc.). In some examples, the gateway may control the SpO2 sensing device to enter either a batch mode or a stream mode. For example, the gateway may transmit such a command based on a command received from a user tier of a sleep monitoring system, as described above. In the batch mode, the device may accumulate SpO2 signals over a predetermined time period before transmitting to the gateway. In the stream mode, the device may transmit SpO2 signals to the gateway in real-time as they are collected.

The method 100 then proceeds to a block 106, where the SpO2 signals are processed at the gateway to generate formatted SpO2 data. This step may involve various data processing techniques to ensure the data is in a suitable format for further analysis. In some implementations, the gateway may format the SpO2 data to generate packets for a particular transport protocol. For instance, MQTT (Message Queuing Telemetry Transport) or similar protocols may be used for packets to the server. The gateway may process the raw SpO2 signals and structure them into a standardized format suitable for MQTT communication.

The gateway may create MQTT packets by first organizing the SpO2 data into a JSON (JavaScript Object Notation) structure. This JSON object may include fields such as a unique device identifier, timestamp, SpO2 value, pulse rate, and any additional relevant metadata. For example, the JSON structure may look like:

{
“device_id”: “SPO2_001”,
“timestamp”: “2023-05-15T10:30:15Z”,
“spo2”: 98,
“pulse_rate”: 72,
“battery_level”: 85,
“mode”: “batch”
}

The gateway may then encapsulate this JSON object into an MQTT message. The gateway may assign an appropriate topic to the message, which may be used for routing and filtering the data on the server side. For instance, the topic structure may follow a hierarchical pattern such as “patient/deviceID/dataType”. In this case, it may be “patient/SPO2_001/vitals”.

The gateway may set the Quality of Service (QoS) level for the MQTT message. For critical health data, the gateway may use QoS level 2 to ensure that the message is received exactly once by the server. For less critical data, QoS level 1 or 0 may be used to balance reliability with efficiency.

In batch mode, the gateway may aggregate multiple SpO2 readings into a single MQTT message to reduce network overhead. The JSON payload may contain an array of readings:

{
“device_id”: “SPO2_001”,
“mode”: “batch”,
“readings”: [
{
“timestamp”: “2023-05-15T22:00:00Z”,
“spo2”: 97,
“pulse_rate”: 68
},
{
“timestamp”: “2023-05-15T22:15:00Z”,
“spo2”: 98,
“pulse_rate”: 70
}
// Additional readings...
]
}

In streaming mode, the gateway may generate MQTT packets more frequently, potentially for each new SpO2 reading, to provide real-time data transmission. Alternatively, in streaming mode, the gateway may use protocols like HTTP Live Streaming (HLS) or Real-Time Streaming Protocol (RTSP) to transmit SpO2 data in real-time. These protocols can be beneficial for continuous, low-latency data transmission in some scenarios.

With HLS, the gateway may segment the SpO2 data stream into small chunks, typically a few seconds long. Each chunk may be encoded as a separate file and added to a playlist. The gateway may create and update a manifest file containing references to these chunks, allowing clients to adapt to network conditions. For RTSP, the gateway may establish a persistent connection with the server, using Real-time Transport Protocol (RTP) for data delivery and RTP Control Protocol (RTCP) for monitoring transmission statistics. This protocol can be suitable for low latency scenarios.

At a block 108, the method 100 involves transmitting the formatted SpO2 data from the gateway to a server for apnea analysis. This transmission may occur through various network protocols, ensuring secure and efficient data transfer.

The method 100 continues at a block 110, where an apnea measure is determined for the cancer patient based on the formatted SpO2 data. In some implementations of block 110, the server may include signal processing techniques to reduce motion artifacts and enhance accuracy. For example, the server may employ adaptive filtering algorithms to isolate the desired SpO2 signal from motion-induced noise. This may involve utilizing accelerometer data, if available, to correlate movement with signal disturbances and subsequently remove these artifacts from the SpO2 readings. The server may also implement machine learning models trained on large datasets to identify and adjust for various types of interference in the SpO2 signal. These models may be capable of recognizing patterns associated with different types of motion, changes in ambient light, or variations in skin perfusion, and modifying the signal accordingly.

In some implementations, the server may perform real-time analysis of the incoming data stream, allowing for prompt feedback to the monitoring device or healthcare providers. This may enable dynamic adjustment of measurement parameters or alert thresholds based on the current quality of the signal and the patient's condition. Additionally, the server may integrate data from multiple sensors or devices to provide a more comprehensive assessment of the patient's physiological state. For instance, it may combine SpO2 data with heart rate variability, respiratory rate, and body temperature to derive more informative health insights.

This step may involve performing feature extraction on the formatted SpO2 data. The feature extraction may include extracting one or more of a sleep architecture feature, a sleep arousal feature, an oxygen saturation feature, or a heart rate feature from the formatted SpO2 data. The feature extraction process may involve various techniques to capture relevant information from the SpO2 data. For sleep architecture features, the system may analyze the SpO2 signal to identify different sleep stages, such as REM and non-REM sleep. This could involve examining the variability and patterns in the SpO2 signal over time. Sleep arousal features may be extracted by detecting sudden changes or fluctuations in the SpO2 signal that could indicate brief awakenings or transitions between sleep stages. These arousals may be characterized by their frequency, duration, and intensity. Oxygen saturation features may include metrics such as the mean SpO2 level, the minimum SpO2 level, the percentage of time spent below certain SpO2 thresholds, and the oxygen desaturation index (ODI). The ODI represents the number of times per hour that the SpO2 level drops by a specified percentage from baseline. Heart rate features may be derived from the SpO2 signal, as many SpO2 sensors can also measure pulse rate. These features could include average heart rate, heart rate variability, and changes in heart rate associated with respiratory events.

In some implementations, various feature extraction techniques may be employed to derive meaningful information from the SpO2 data for sleep apnea analysis. For sleep architecture features, time-frequency analysis techniques such as wavelet transforms or short-time Fourier transforms may be applied to the SpO2 signal. These methods may help identify characteristic patterns associated with different sleep stages. For example, the system may detect changes in signal variability that correspond to transitions between REM and non-REM sleep.

Sleep arousal features may be extracted using change point detection algorithms. These algorithms may identify sudden shifts in the statistical properties of the SpO2 signal, which may indicate brief awakenings or sleep stage transitions. In some cases, adaptive thresholding techniques may be used to detect rapid increases in SpO2 levels that often accompany arousals.

For oxygen saturation features, statistical measures may be computed over different time windows. The system may calculate rolling averages, standard deviations, and percentiles of the SpO2 signal. To determine the oxygen desaturation index (ODI), peak detection algorithms may be applied to identify significant drops in SpO2 levels. The frequency and magnitude of these desaturation events may be quantified to characterize the severity of potential sleep apnea.

Heart rate features may be extracted using peak detection algorithms to identify individual heartbeats from the SpO2 waveform. Time domain measures such as the standard deviation of NN intervals (SDNN) or the root mean square of successive differences (RMSSD) may be computed to quantify heart rate variability. In some implementations, frequency domain analysis techniques like the Lomb-Scargle periodogram may be applied to assess the balance between sympathetic and parasympathetic nervous system activity.

In some cases, more advanced feature extraction techniques may be employed. For instance, recurrence quantification analysis may be used to capture nonlinear dynamics in the SpO2 signal. This method may reveal subtle patterns that are not apparent through traditional linear analysis. Additionally, entropy-based measures such as sample entropy or approximate entropy may be computed to quantify the complexity and regularity of the SpO2 time series.

The system may also utilize dimensionality reduction techniques like principal component analysis (PCA) or t-distributed stochastic neighbor embedding (t-SNE) to identify informative features from the high-dimensional SpO2 data. These methods may help in visualizing patterns and clusters in the data that could be indicative of different sleep apnea severities or types.

In some implementations, the feature extraction process may be adaptive, adjusting its parameters based on the quality of the incoming signal or the specific characteristics of the patient. For example, the window sizes used for computing rolling statistics may be dynamically adjusted based on the observed variability in the SpO2 signal.

The apnea measure output by the model may be a continuous score indicating the severity of sleep apnea, or it could be a categorical classification (e.g., no apnea, mild apnea, moderate apnea, severe apnea). The choice between these approaches may depend on the specific requirements of the monitoring system and the preferences of healthcare providers.

An example apnea measure is the progression of the Apnea-Hypopnea Index (AHI) in cancer patients undergoing treatment. A mixed linear model may be used that leverages longitudinal cancer patient data collected from the device, which includes multiple patient visits, providing a comprehensive view of dynamic changes in physiological parameters over time. The analysis may estimate the change rate of AHI over visiting time, providing insights for proactive intervention. As an example, apnea categories may be employed as the independent variable with AHI≥5 categorized as apnea and AHI<5 categorized as non-apnea. Changes in AHI may be quantified as the relative change between two consecutive measurements (Δ AHI), calculated using data collected from the device as:

$$Δ A H I = \frac{A H {I}_{t + 1} - A H {I}_{t}}{A H {I}_{t}}$$

Where ΔAHI denotes the change rate of AHI between two measurements, AHIₜ represents the AHI value at time t, and the subscripts denote consecutive measurements.

To determine the apnea measure, a mixed linear model may be applied to the extracted features. The mixed linear model may be represented as:

$$Y = X β + Z μ + ϵ$$

Where Y is the response variable (apnea measure), X is the design matrix for fixed effects, β is the vector of fixed effects coefficients, Z is the design matrix for random effects, p is the vector of random effects, and ε is the error term. Here, Y is an N×1 column vector representing the outcome variable, X is an N×p matrix of p predictor variables, β is a p×1 column vector of fixed-effects regression coefficients, Z is an N× qJ design matrix for q random effects and J groups, μ is a qJ×1 vector of random effects for J groups, and E is an N×1 column vector of residuals. As an example, predictors may include BMI, sleep architecture, arousals, oxygen saturation, and heart rate.

In this example, X may be an N×p matrix where each row corresponds to a sleep session and each column represents a predictor variable. For instance, for N sleep sessions and p=5 predictor variables, the X matrix may look like:

$$X = [ \begin{matrix}B M {I}_{1} & S {A}_{1} & A {R}_{1} & O {S}_{1} & H {R}_{1} \\ B M {I}_{2} & S {A}_{2} & A {R}_{2} & O {S}_{2} & H {R}_{2} \\ ⋮ & ⋮ & ⋮ & ⋮ & ⋮ \\ B M {I}_{N} & S {A}_{N} & A {R}_{N} & O {S}_{N} & H {R}_{N}\end{matrix} ]$$

Where BMI represents Body Mass Index, SA represents Sleep Architecture (e.g., percentage of time in REM sleep), AR represents Arousal Index (number of arousals per hour), OS represents Oxygen Saturation (e.g., mean SpO2 level), and HR represents Heart Rate (e.g., mean heart rate during sleep). Each row in this matrix may correspond to a single sleep session, with the subscript indicating the session number. For example, BMI₁ represents the BMI value for the first sleep session, SA₂ represents the sleep architecture measure for the second sleep session, and so on.

In some implementations, the X matrix may include additional predictor variables such as age, gender, or specific cancer treatment information. The number and type of predictor variables may be adjusted based on the available data and the specific requirements of the sleep apnea analysis for cancer patients.

The mixed linear model may incorporate both fixed effects, which are constant across all patients, and random effects, which can vary between patients. Fixed effects might include factors such as age, gender, or cancer type, while random effects could account for individual patient variability that is not explained by the fixed effects.

In determining the number J of groups based on cancer patient training data, various approaches may be employed. One method may involve using clustering algorithms to identify natural groupings within the patient population. For example, k-means clustering or hierarchical clustering techniques may be applied to the training data, with the number of clusters determined through methods such as the elbow method or silhouette analysis.

Groups may be defined based on clinically relevant factors such as cancer type, stage, or treatment modality. For instance, patients could be grouped by primary cancer site (e.g., lung, breast, colorectal) or treatment regimen (e.g., chemotherapy, radiation therapy, immunotherapy). A combined approach may use unsupervised learning to identify potential groupings, followed by refinement and validation by clinical experts to ensure relevance and interpretability. Cross-validation techniques like k-fold may be used to assess the stability and generalizability of the grouping structure across different subsets of the training data.

The coefficients and random effects for the mixed linear model may be determined through training on cancer patient data. This data may come from public datasets or gathered datasets specific to cancer patients with sleep disorders. The training process may involve techniques such as maximum likelihood estimation or restricted maximum likelihood estimation to optimize the model parameters.

In some implementations, a logarithmic transformation may be applied to the output of the mixed-linear model to determine the final apnea measure. This transformation may help in normalizing the data and improving the model's performance. The logarithmic transformation applied to the output of the mixed-linear model to determine the final apnea measure may be represented as:

$$A = log ( Y + 1 )$$

Where A is the final apnea measure and Y is the output from the mixed-linear model. The addition of 1 inside the logarithm may help avoid undefined values when Y is zero or close to zero. This transformation may compress the range of values, potentially making the distribution of the apnea measure more normal and reducing the impact of extreme values. To address the nonlinear relationship between AHI change rates and predictor variables, a logarithmic transformation may be applied to the outcome variable, as expressed by:

$${Y}^{′} = log ( Y + 1 )$$

Where Y′ represents the transformed value of the change rate of AHI between two measurements, and Y denotes the change rate of AHI.

The transformed variable Y′ may be used to estimate the progression of AHI over various time scales, including days, weeks, and months. By applying this transformation to the change rate of AHI between consecutive measurements, the system may track and analyze the evolution of sleep apnea severity in cancer patients over different temporal resolutions. The following examples illustrate longitudinal data analysis approaches for tracking the progression of AHI in cancer patients over different time scales. For daily progression, Y′ may represent the day-to-day changes in AHI, potentially revealing short-term fluctuations in sleep apnea severity that may be associated with factors such as daily variations in medication effects or immediate responses to treatment interventions. When examining weekly progression, Y′ may capture trends that emerge over a series of days, which may be useful for assessing the impact of weekly treatment cycles or identifying patterns related to work-rest schedules. For monthly progression, Y′ may provide insights into longer-term changes in AHI, which may be particularly relevant for evaluating the overall effectiveness of cancer treatments on sleep apnea symptoms or identifying seasonal variations in sleep quality. By analyzing Y′ across these different time scales, healthcare providers may gain a comprehensive understanding of how sleep apnea progresses in cancer patients throughout their treatment journey. This multi-scale approach may allow for the detection of both acute changes and gradual trends in sleep apnea severity, potentially enabling more timely and targeted interventions.

In addition to the logarithmic transformation, other data transformations or normalization techniques may be applied to improve the model's performance. These could include standardization (scaling to zero mean and unit variance) or min-max scaling of input features.

The training process for the mixed linear model may involve cross-validation techniques to ensure the model generalizes well to new data. This could include k-fold cross-validation or leave-one-out cross-validation, depending on the size of the available dataset. The coefficients and random effects for the mixed linear model may be determined through training on cancer patient data. This data may come from public datasets or gathered datasets specific to cancer patients with sleep disorders. The training process may involve techniques such as maximum likelihood estimation or restricted maximum likelihood estimation to optimize the model parameters.

The collected data may be used to train a mixed linear model to predict the progression of AHI scores over patient visits. This training process may involve feature engineering, model fitting, and optimization techniques. To evaluate the trained model, a separate dataset of longitudinal data from additional cancer patients with OSA may be used. The model's effectiveness in predicting AHI scores may be assessed using several performance metrics, which could include accuracy, precision, recall, and F1-score.

Finally, at a block 112, the method 100 concludes with providing a longitudinal progression of the apnea measure over the plurality of sleep sessions. This longitudinal progression may be presented through a user service, allowing healthcare professionals to track changes in the patient's sleep patterns and apnea conditions over time.

In some implementations, the longitudinal progression of the apnea measure may be presented as a graph of changes in AHI over time. This graphical representation may provide healthcare professionals with a visual tool to track and analyze the patient's sleep apnea condition throughout the course of cancer treatment. The graph may display the AHI values on the y-axis and time on the x-axis, allowing for easy visualization of trends and patterns in the patient's apnea severity. Each data point on the graph may represent an AHI measurement from a sleep session, with connecting lines between points to illustrate the progression.

The longitudinal progression of the apnea measure may be presented as a graph displaying AHI values over time, allowing healthcare professionals to track trends in the patient's sleep apnea condition. The graph may incorporate features such as color-coding for apnea severity categories, adjustable time scales, error bars or confidence intervals, interactive elements for additional data display, and overlay options for treatment milestones or other relevant parameters. This comprehensive visualization, potentially integrated into a broader dashboard, may facilitate informed decision-making and personalized patient care strategies.

While the mixed linear model provides a robust approach for determining the apnea measure, alternative machine learning models may also be applied. For example, a reservoir computer may be used as an alternative approach. A reservoir computer is a type of recurrent neural network that may be particularly suited for time-series data such as SpO2 signals. The reservoir computer may consist of a large, fixed, randomly connected hidden layer (the reservoir) and a simple readout layer that is trained on the task at hand. This approach may offer advantages in terms of computational efficiency and the ability to capture complex temporal dynamics in the SpO2 data.

The method 100 for monitoring SpO2 signals provides a comprehensive approach to sleep monitoring in cancer patients, offering valuable insights into sleep patterns and potential apnea conditions. By leveraging advanced data processing techniques and machine learning models, this method may contribute to improved patient care and treatment outcomes.

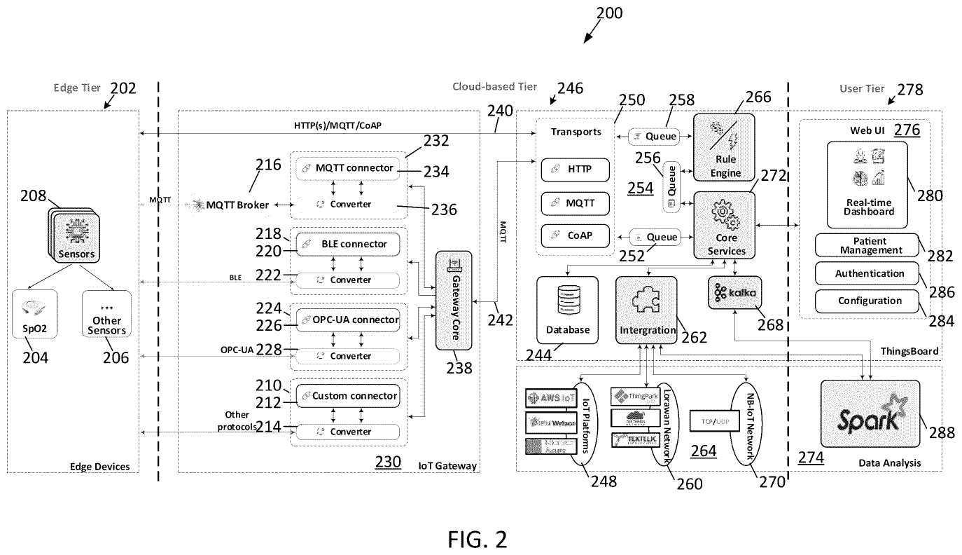

FIG. 2 illustrates an example system 200 for monitoring SpO2 signals from cancer patients. For example, the system 200 may perform method 100 of FIG. 1 or other method described herein. The system architecture 200 may comprise three main tiers: an edge device 202, a cloud-based tier, and a user tier 278. At the Edge tier, sensor devices may acquire and transmit data through protocols such as HTTP, MQTT, CoAP, etc. These data may be directed via an IoT Gateway to the central cloud server, where they may undergo transformation and storage.

The Cloud-based Tier may encompass various modules. Using ThingsBoard as an example platform, these modules may include a rule chain engine for data transformation and basic data analytics, applying rule-based logic to filter or transform incoming and ongoing data. The data may then proceed to core services for storage in a database and an advanced data analytics module for further processing. A Queue Message Module, potentially supporting stream management services like Kafka, RabbitMQ, AWS SQS, and Google Pub/Sub, may facilitate data handling. A database may store telemetry and device configuration data, with potential flexibility for configuring databases such as MySQL and PostgreSQL via the Rule Chain Engine. Kafka may serve as a bridge, potentially connecting to Spark Streaming for real-time data processing. A data analytics module may retrieve data from Kafka topics, organize them into discrete streams, and initiate processing.

The edge device 202 may include a pulse oximeter for collecting SpO2 signals from cancer patients. In some cases, the edge device 202 may transmit the collected SpO2 signals to other components of the system architecture 200 for further processing and analysis.

The edge tier includes a gateway to connect to the edge device 202 via a local connection. The gateway may receive SpO2 signals from the edge device 202, process the SpO2 signals to generate formatted SpO2 data, and transmit the formatted SpO2 data for further analysis. In some cases, the cloud-based tier may include an analysis service 274 that executes a model 288 to monitor apnea of cancer patients based on the formatted SpO2 data.

The user tier 278 may provide a platform for patient management and data visualization. In some cases, the user tier 278 may allow healthcare professionals to access and interpret the results of the apnea monitoring performed by the analysis service 274.

The edge device 202 may include various components for collecting and processing physiological data from cancer patients. In some cases, the edge device 202 may comprise a SpO2 sensor 204 for measuring oxygen saturation levels in the blood. The edge device 202 may also include other sensors 206 for collecting additional physiological data.

Collectively, these sensors 208 may form the data collection core of the edge device 202. In addition to the SpO2 sensor 204, the edge device 202 may incorporate various other sensors 206 to provide a comprehensive view of the patient's physiological state during sleep. These additional sensors may include:

The edge device 202 may incorporate various sensors 208 to provide a comprehensive view of the patient's physiological state during sleep. These may include: 1) Electrocardiogram (ECG) sensors for monitoring heart electrical activity, detecting cardiac arrhythmias, assessing heart rate variability, and providing insights into autonomic nervous system function during different sleep stages. 2) A pulsemeter for continuous pulse rate measurement, complementing SpO2 readings and providing additional cardiovascular function information. 3) Respiratory effort sensors such as chest and abdominal bands to measure thorax and abdomen expansion/contraction during breathing, helping distinguish between obstructive and central sleep apnea events. 4) Accelerometers to detect body movement and position changes, aiding in identifying sleep-wake cycles, assessing sleep quality, and detecting periodic limb movements. 5) Temperature sensors to monitor body temperature fluctuations, potentially providing insights into circadian rhythms and sleep-wake patterns. 6) Electroencephalogram (EEG) sensors to measure brain activity for sleep stage classification. 7) Electromyogram (EMG) sensors to measure muscle activity, particularly in chin and leg areas, useful for detecting REM sleep and assessing conditions like REM sleep behavior disorder. 8) Electrooculogram (EOG) sensors to track eye movements, useful for identifying REM sleep periods and certain sleep disorders. 9) Microphones to detect and record snoring or abnormal breathing sounds indicative of sleep-disordered breathing. 10) Ambient light sensors to measure sleep environment light levels, providing context for sleep-wake patterns and potential environmental disturbances. 11) Humidity sensors to monitor sleep environment humidity, providing additional context for interpreting respiratory data and assessing overall sleep quality. By incorporating some or all of these sensors, the edge device 202 may provide a more comprehensive assessment of the patient's sleep patterns and physiological state. The combination of data from multiple sensors may enable more accurate detection of sleep disorders and a better understanding of the complex interactions between various physiological systems during sleep. Some or all of these sensors 208 may form the data collection core of the edge device 202.

In some implementations, the edge device 202 may include an analog front-end for processing SpO2 signals from the spo2 sensor 204. The analog front-end may amplify and filter the raw SpO2 signals to improve signal quality and reduce noise. The analog front-end may include an analog to digital converter (ADC) to transform the analog SpO2 signals into digital data. This ADC may sample the amplified and filtered SpO2 waveform at a predetermined rate, converting the continuous analog signal into discrete digital values. In some implementations, the ADC may have a resolution of 12 to 16 bits, allowing for precise digitization of the SpO2 signal. The sampling rate of the ADC may be adjustable, potentially ranging from 100 Hz to 1 kHz, to accommodate different monitoring requirements and power consumption considerations. The digitized SpO2 data may then be passed to the microcontroller for further processing and analysis.

The edge device 202 may also comprise a microcontroller to further process the SpO2 signals. In some cases, the microcontroller may convert the analog signals from the spo2 sensor 204 into digital data suitable for transmission. The microcontroller may also perform initial data analysis or compression to optimize data transmission.

To facilitate communication with other components of the system 200, the edge device 202 may include various protocol interfaces. In some implementations, the edge device 202 may include a protocol interface 210 with a connector 212 and a protocol converter 214. The protocol interface 210 may enable the edge device 202 to communicate using different protocols as desired.

For Bluetooth Low Energy (BLE) communication, the edge device 202 may include a BLE interface 218 with a BLE connector 220 and a BLE converter 222. This may allow for short-range, low-power communication with nearby devices. In some cases, the edge device 202 may also include an OPC interface 224 with an OPC connector 226 and an OPC converter 228. This may enable communication using the OPC (Open Platform Communications) protocol, which may be useful in certain industrial or healthcare settings. For MQTT (Message Queuing Telemetry Transport) communication, the edge device 202 may include an MQTT interface 232 with an MQTT connector 234 and an MQTT converter 236. The edge device 202 may also include an MQTT broker 216 to manage MQTT message routing.

The edge device 202 may communicate with a gateway device 230 through a gateway interface 238. This interface may allow the edge device 202 to transmit collected and processed data to other components of the system 200 for further analysis and storage.

In some implementations, the edge device 202 may operate in different modes to optimize data collection and transmission. In a batch mode, the edge device 202 may accumulate data over a predetermined period before transmitting it to the gateway device 230. This mode may be useful for conserving power and network resources. In a streaming mode, the edge device 202 may transmit data in real-time as it is collected, which may be beneficial for immediate monitoring and analysis.

The compact design of the edge device 202 may be optimized for patient comfort and ease of use. In some cases, the edge device 202 may have a PCB enclosure with dimensions of approximately 64 mm in width, 32 mm in height, and 93 mm in length. This compact form factor may allow for unobtrusive monitoring of cancer patients during sleep sessions.

The cloud-based tier of the system 200 may connect to the edge device 202 through a transport layer 240 and communication link 242. This tier may contain a data manager 246 that includes a storage system 244 to store SpO2 data. The data manager 246 may connect to the edge device 202 via the gateway device 230. The data manager 246 may include a communication interface 250 that handles the connections 242, 240. The output queue 252, work queue 256, and input queue 258 may be managed by the components that use the queues or could be managed by service manager 272. In some implementations, the queues may be utilized to efficiently manage data flow and processing within the system. The input queue 258 may serve as a buffer for incoming data from the edge device 202 or other data sources. As SpO2 signals and other physiological data arrive, they may be temporarily stored in the input queue 258, allowing the system to handle bursts of incoming data without overwhelming the processing capabilities. This queue may help ensure that no data is lost during periods of high data influx.

The work queue 256 may be used to manage tasks to be processed by the system. These tasks may include data analysis, feature extraction, or model execution. As data becomes available for processing, it may be moved from the input queue 258 to the work queue 256. The work queue 256 may prioritize tasks based on various factors such as urgency, computational requirements, or user-defined priorities. This queue may help optimize resource allocation and ensure that tasks are processed in a timely manner. The output queue 252 may store processed results and formatted data that are ready for transmission to other components of the system, such as the user tier 278 or external services. Data in the output queue 252 may include analyzed SpO2 data, apnea measures, or other derived metrics. This queue may help manage the flow of outgoing data, ensuring that results are delivered efficiently and in the correct order.

The cloud-based tier may include an integration interface 262 that enables data integrations 264 with external systems or services. This may allow for seamless data exchange and interoperability with other healthcare information systems or analytical tools. In some cases, the cloud-based tier may incorporate various platforms and systems to enhance data processing and connectivity. For example, an IoT platform 248 may be integrated to facilitate device management and data collection. A LoRaWAN system 260 may be included to support long-range, low-power communication with compatible devices. The cloud-based tier may also feature a narrowband IoT platform 270 for efficient, wide-area cellular connectivity.

A rule service 266 may be implemented to process data and communicate with a service manager 272. In some implementations, the rule service 266 may utilize a rule engine for data transformation and basic analytics. For example, a ThingsBoard Rule Chain Engine may be employed to define and execute rules for data processing and routing. The rule service 266 may utilize the ThingsBoard Rule Chain Engine to perform feature extraction on the incoming SpO2 data, generating feature vectors for use in the model 288. The rule engine may apply various data processing and analysis techniques to extract relevant features from the raw SpO2 signals and other physiological data collected by the edge device 202. For example, the rule engine may implement the following feature extraction processes: 1. Time-domain features: The rule engine may calculate statistical measures from the SpO2 time series data, such as mean, median, standard deviation, and percentiles. These features may provide insights into the overall oxygen saturation levels and their variability over time. 2. Frequency-domain features: The rule engine may apply Fast Fourier Transform (FFT) or other spectral analysis techniques to the SpO2 signal, extracting features such as dominant frequencies and power spectral density. These features may help identify periodic patterns in oxygen saturation levels that could be indicative of sleep apnea events. 3. Oxygen desaturation events: The rule engine may implement algorithms to detect and quantify oxygen desaturation events, such as the number of events per hour, average duration of events, and the magnitude of desaturation. These features may be useful for assessing the severity of sleep apnea. 4. Heart rate variability: If heart rate data is available from the pulsemeter or ECG sensors, the rule engine may calculate heart rate variability metrics such as RMSSD (Root Mean Square of Successive Differences) or pNN50 (proportion of NN intervals differing by more than 50 ms). These features may provide information about autonomic nervous system function during sleep. 5. Respiratory features: For data from respiratory effort sensors, the rule engine may extract features such as breathing rate, inspiratory time, and expiratorytime. These features may help distinguish between different types of sleep apnea events. 6. Movement-related features: Using data from accelerometers, the rule engine may derive features related to body position changes and limb movements, which may be relevant for identifying sleep disturbances. 7. Sleep stage indicators: If EEG data is available, the rule engine may implement algorithms to extract features indicative of different sleep stages, such as spectral power in specific frequency bands (e.g., delta, theta, alpha). The rule engine may combine these extracted features into feature vectors, which may then be passed to the model 288 for further analysis and apnea measure determination. The rule engine may also apply normalization or scaling techniques to ensure that all features are on a comparable scale before being input into the model. In some cases, the rule engine may adapt its feature extraction processes based on the available sensor data and the specific requirements of the model 288. This flexibility may allow the system to optimize its performance for different patient populations or monitoring scenarios.

The system 200 may incorporate an event stream service 268 to manage data streaming between components. In some cases, Apache Kafka may be used as the event stream service 268, providing a distributed streaming platform for building real-time data pipelines and streaming applications.

The analysis service 274 may play a role in processing and analyzing the collected SpO2 data. This service may utilize advanced machine learning techniques to extract meaningful insights from the data. In some implementations, the analysis service 274 may employ a machine learning platform such as Apache Spark for data processing and analysis. Apache Spark's distributed computing capabilities may allow for efficient processing of large volumes of SpO2 data in real-time or batch mode.

The model 288 within the analysis service 274 may be designed to perform various analytical tasks. For example, the model 288 may be trained to detect anomalies in SpO2 readings, predict potential sleep apnea events, or classify sleep stages based on the collected data. The model 288 may utilize techniques such as supervised learning, unsupervised learning, or deep learning depending on the specific analytical requirements.

In some cases, the model 288 may incorporate feature engineering techniques to extract relevant features from the raw SpO2 data. These features may include statistical measures (e.g., mean, standard deviation, percentiles), frequency domain features (e.g., spectral power in specific frequency bands), or time-domain features (e.g., signal variability, trend analysis).

The analysis service 274 may also include capabilities for model training, validation, and updating. This may involve periodically retraining the model 288 with new data to improve its accuracy and adapt to changing patient conditions. The service may implement techniques such as cross-validation and hyperparameter tuning to optimize the model's performance.

To support data storage and retrieval, the cloud-based tier may utilize a database system within the storage system 244. This database may be designed to efficiently store and manage telemetry data from the SpO2 sensors as well as device configuration information. In some implementations, a NoSQL database such as Apache Cassandra may be used to handle the high-volume, time-series nature of the SpO2 data. The database may store various types of data, including raw SpO2 measurements, derived metrics such as heart rate and oxygen saturation levels, patient profile information like age, gender, and medical history, and model parameters specific to each patient. Additionally, the database may contain aggregated statistics, trend analyses, and historical comparisons of sleep quality metrics. It may also store environmental data collected during sleep sessions, such as room temperature and ambient noise levels, to provide context for the physiological measurements. The system may also retain metadata about each sleep session, including start and end times, device settings, and any notes or observations from healthcare providers. This comprehensive data storage approach may enable more nuanced analysis and personalized care strategies for cancer patients undergoing treatment.

The cloud-based tier components may work together to provide a comprehensive platform for SpO2 data collection, storage, processing, and analysis. The combination of real-time data streaming, advanced analytics, and flexible data storage may enable healthcare providers to gain valuable insights into patients' sleep patterns and potential sleep disorders. The user tier 278 of the system 200 may provide a platform for healthcare professionals to interact with patient data and manage monitoring activities. A component of the user tier 278 may be a user interface 276, which may offer various functionalities for data visualization and system control.

Within the user interface 276, a dashboard 280 may be implemented to provide an overview of patient data, including the apnea measure over time. The dashboard 280 may present this information in a graphical format, allowing healthcare professionals to easily track changes in a patient's condition across multiple sleep sessions. In some cases, the dashboard 280 may include interactive elements that enable users to adjust the time scale or focus on specific data points for more detailed analysis.

The user interface 276 may also include features for patient management 282, allowing healthcare professionals to organize and access patient records efficiently. This may involve tools for adding new patients, updating existing records, and categorizing patients based on various criteria such as cancer type or treatment stage. A configuration 284 component within the user interface 276 may allow users to customize system settings and preferences. This may include options for adjusting data display formats, setting alert thresholds for apnea measures, or configuring data export settings for integration with other healthcare information systems.

To ensure data security and privacy, the user interface 276 may incorporate an authentication 286 mechanism. This may require healthcare professionals to log in with secure credentials before accessing patient data or system controls. In some cases, the authentication 286 system may support role-based access control, allowing administrators to define different levels of access for various user types.

The user interface 276 may provide functionality for healthcare practitioners to control the edge device 202 remotely. For example, a healthcare professional may use the user interface 276 to instruct the edge device 202 to enter a streaming mode. In the streaming mode, the edge device 202 may transmit real-time pulse oximeter data, which may be particularly useful during patient visits or checkups. This real-time data streaming may allow healthcare professionals to observe immediate changes in a patient's SpO2 levels and make on-the-spot assessments.

In some implementations, the user interface 276 may offer tools for generating reports based on the collected data. These reports may summarize metrics, highlight trends in apnea measures, and provide insights that may assist healthcare professionals in making informed decisions about patient care and treatment adjustments. The user tier 278 may be designed with a focus on usability and efficiency, aiming to streamline the process of monitoring and managing cancer patients' sleep patterns. By providing intuitive access to comprehensive patient data and system controls, the user tier 278 may support healthcare professionals in delivering more personalized and effective care to cancer patients experiencing sleep-related issues.

FIG. 3A and FIG. 3B illustrate orthogonal views of a sensor device 300. FIG. 3A shows an internal view of the sensor device 300 with an enclosure housing 302 opened to reveal internal components. The circuit board 306 may be mounted within the enclosure housing 302 and may contain a microcontroller 310 and an analog front-end 312. A battery 308 may be positioned adjacent to the circuit board 306 to provide power. A port 304 may be integrated into the circuit board 306 for connection to a sensor cable 316.

FIG. 3B shows an external view of the sensor device 300 during operation. The enclosure housing 302 may be shown connected to a SpO2 sensor 314 via the sensor cable 316. The SpO2 sensor 314 may be configured to attach to a finger for measuring oxygen saturation levels. The sensor cable 316 may provide the electrical connection between the SpO2 sensor 314 and the internal components housed within the enclosure housing 302.

The sensor device 300 may be designed to collect SpO2 signals from a cancer patient undergoing treatment over multiple sleep sessions. The SpO2 sensor 314 may gather analog sensor data by emitting light at two different wavelengths, typically red and infrared, through a translucent part of the patient's body, such as a fingertip or earlobe. As the light passes through the body tissue, some of the light may be absorbed by blood and soft tissues, depending on the concentration of oxygenated and deoxygenated hemoglobin.

The enclosure housing 302 may be designed with compact dimensions to enhance portability and minimize disruption to the patient's sleep environment. In some implementations, the housing may measure approximately 80 mm in length, 50 mm in width, and 20 mm in height. These compact dimensions may allow the sensor device 300 to be easily placed under a pillow or on a bedside table without causing discomfort or interfering with the patient's normal sleep positioning.

The small form factor of the sensor device 300 may offer several benefits in terms of usability and patient comfort. Its compact size may allow for discreet placement in the sleep environment, potentially reducing any psychological impact on the patient's sleep quality that might arise from the presence of a more conspicuous monitoring device. For example, the device may be tucked under the edge of a pillow or placed on a nearby nightstand, remaining out of sight while still maintaining optimal signal reception.

The sensor device 300 may be designed to operate automatically without requiring active user attention. Once set up and activated, the device may continuously monitor and record SpO2 data throughout the night without any need for patient intervention. This hands-off approach may be particularly beneficial for cancer patients who may already be dealing with fatigue or discomfort from their treatments, as it minimizes the additional burden of managing a monitoring device.

In some implementations, the sensor device 300 may be operated entirely under the control of a remote practitioner's system. For instance, a healthcare professional may use the user interface 276 to remotely configure and control the sensor device 300. The practitioner may be able to initiate monitoring sessions, adjust sampling rates, or switch between batch and streaming modes without requiring any action from the patient.

As an example, a remote practitioner may schedule the sensor device 300 to activate automatically at a predetermined time each night. The device may then collect SpO2 data throughout the night, operating in batch mode to conserve battery life. In the morning, the practitioner's system may send a command to the sensor device 300 to transmit the collected data for analysis. If the analysis reveals any concerning patterns, the practitioner may remotely switch the device to streaming mode for real-time monitoring during the next sleep session, all without requiring the patient to make any adjustments to the device.

This remote operation capability may enhance the efficiency of sleep monitoring for cancer patients, allowing for continuous, adaptive monitoring without increasing the patient's care management burden. It may also enable healthcare providers to make timely adjustments to monitoring protocols based on the patient's changing condition or treatment regimen.

The analog front-end 312 may process the raw SpO2 signals from the SpO2 sensor 314. The analog front-end 312 may amplify and filter the signals to improve signal quality and reduce noise. In some cases, the analog front-end 312 may include an analog-to-digital converter (ADC) to transform the analog SpO2 signals into digital data. The ADC may sample the amplified and filtered SpO2 waveform at a predetermined rate, converting the continuous analog signal into discrete digital values.

The microcontroller 310 may further process the digitized SpO2 data. In some cases, the microcontroller 310 may calculate the ratio of oxygenated to deoxygenated hemoglobin in the arterial blood. This ratio may then be converted into a SpO2 percentage, which represents the oxygen saturation of the blood. The microcontroller 310 may also provide additional information, such as heart rate, by analyzing the frequency of the pulsatile signal.

The battery 308 may power the sensor device 300, allowing for portable and continuous monitoring of the patient's SpO2 levels during sleep sessions. The compact design of the sensor device 300, facilitated by the enclosure housing 302, may allow for unobtrusive monitoring of cancer patients during sleep sessions.

In some cases, the microcontroller 310 may transmit the processed SpO2 signals to the gateway device through a wireless communication protocol. The port 304 may facilitate this communication, potentially supporting various protocols such as Bluetooth Low Energy (BLE), Wi-Fi, or other suitable wireless technologies.

The sensor device 300 may operate in different modes to optimize data collection and transmission. In a batch mode, the sensor device 300 may accumulate data over a predetermined period before transmitting the data to the gateway device. In a streaming mode, the sensor device 300 may transmit data in real-time as the data may be collected, which may be beneficial for immediate monitoring and analysis.

By integrating these components, the sensor device 300 may provide a comprehensive solution for collecting and processing SpO2 data from cancer patients over multiple sleep sessions. The compact and portable design may allow for continuous monitoring while minimizing disruption to the patient's sleep, potentially providing valuable insights into sleep patterns and potential apnea conditions during cancer treatment.

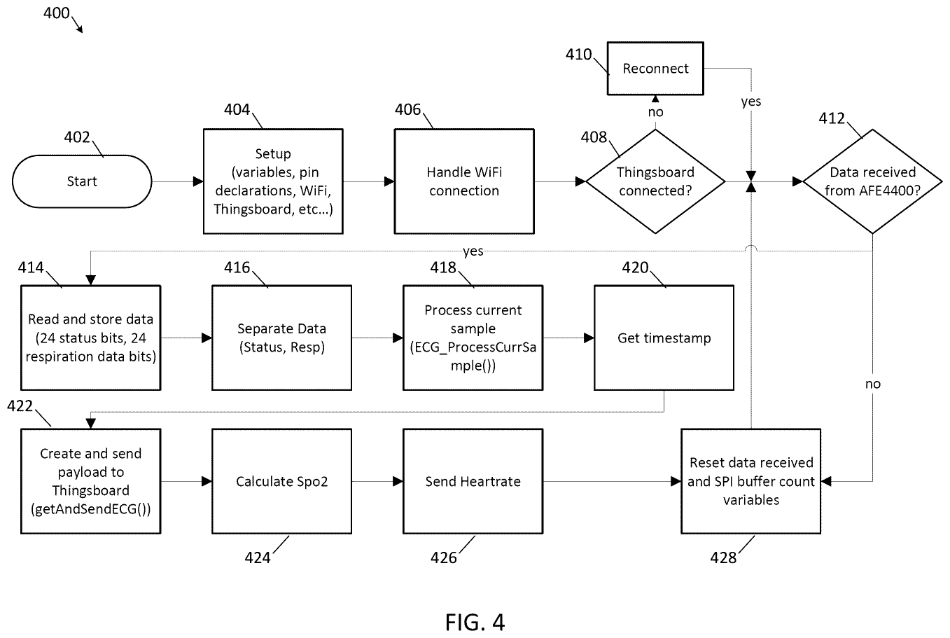

FIG. 4 illustrates a flowchart of a method 400 for processing and transmitting SpO2 data. The method 400 may be described with respect to the operations of a microcontroller in a sensor device, such as the microcontroller 310 in the sensor device 300, as it interacts with other components of the system. This method may illustrate how the microcontroller processes and transmits SpO2 data collected from a patient, coordinating with various hardware and software elements to ensure efficient data handling and communication.

In some implementations, the microcontroller may execute the steps of method 400 to manage the collection, processing, and transmission of SpO2 data. The method may involve interactions between the microcontroller and other components such as the analog front-end, wireless communication modules, and external systems like the gateway device and cloud-based services.

The microcontroller may orchestrate the flow of data from the initial signal acquisition through to the transmission of processed data to other parts of the system. This may include managing the analog-to-digital conversion of SpO2 signals, performing initial data processing, handling wireless connectivity, and coordinating data transmission protocols.

The method 400 may begin at a block 402 with a start operation. At block 404, the system 200 may perform setup operations including variable and pin declarations, and WiFi and IoT platform configurations. For example, the IoT platform may be Thingsboard. The method 400 may proceed to a block 406 where the system 200 may handle the WiFi connection.

At a decision block 408, the method 400 may determine if the IoT platform is connected. If not connected, the flow may move to a block 410 for reconnection attempts. If connected, the flow may proceed to a decision block 412 to check if data is received from an analog front-end (AFE).

When data is received, the method 400 may move to a block 414 where the system 200 may read and store data (24 status bits, 24 respiration data bits). At a block 416, the data may be separated into Status and Response components. The method 400 may then proceed to a block 418 to process the current sample using ECG_ProcessCurrSample( ).

At a block 420, the method 400 may get a timestamp for the data. A block 422 may involve creating and sending a payload to the IoT platform using getAndSendECG( ). The method 400 may then move to a block 424 to calculate SpO2 values and a block 426 to send heartrate data.

If at the decision block 412 no data is received from AFE, or after completing the data transmission steps, the method 400 may proceed to a block 428 where the system 200 may reset data received and SPI buffer count variables. The process may then return to the decision block 408 to continue monitoring for IoT platform connectivity.

In some cases, the gateway device 230 may control the sensor device 300 to enter either a batch mode or a stream mode for data transmission. The batch mode may allow the sensor device 300 to accumulate SpO2 signals over a predetermined time period before transmitting to the gateway device 230. This mode may provide long term longitudinal data collection, potentially supporting extended time between battery 308 charges, such as days or weeks.

In the stream mode, the sensor device 300 may transmit SpO2 signals to the gateway device 230 in real-time as they are collected. This real-time data transmission may provide functionality similar to pulse oximeters in clinical environments. The stream mode may be useful for immediate monitoring and analysis of a patient's SpO2 levels.

The method 400 may incorporate these different modes of operation, allowing for flexible data collection and transmission based on the specific monitoring requirements and power considerations. For example, in batch mode, the method 400 may accumulate data at the block 414 over an extended period before proceeding to the data transmission steps. In stream mode, the method 400 may proceed through the data processing and transmission steps more frequently, potentially after each data collection cycle.

By incorporating both batch and stream modes, the method 400 may provide a versatile approach to SpO2 data processing and transmission, catering to various monitoring scenarios and power management requirements in cancer patient care.

FIG. 5 illustrates an example of patient use and doctor interaction with the sleep monitoring system. The figure depicts a sequence diagram 500 showing the data flow and interactions between a patient 502, doctor 504, hardware components (analog front-end (AFE) 506 and microcontroller 508), and software components (data manager 510 and mixed linear model 512) in the sleep monitoring system.

The sequence begins with a loop structure where the patient 502 initiates data collection through a data measurement 514 process using the analog front-end 506. The analog front-end 506 processes this data and sends digitized measurement 516 to the microcontroller 508. The microcontroller 508 then initializes WiFi connectivity through a network initialization 518 call, followed by establishing a connection activation 520 and receiving a status check 522.

Once the connection is established, the microcontroller 508 sends the processed data through a data transmission 524 to the data manager 510, which forwards it via data processing transmission 526 to the mixed linear model 512 for analysis. The mixed linear model 512 performs data analysis 528 and returns the results 530 to the data manager 510.

In parallel, there is a separate interaction where the doctor 504 can send a request message 532 to the data manager 510 for patient information. The data manager 510 then returns the requested patient information through a request response 534.

The system may operate in two modes: batch mode and streaming mode. In batch mode, the microcontroller 508 may accumulate data over a predetermined period before transmitting it to the data manager 510. This mode may be useful for conserving power and network resources. In streaming mode, the microcontroller 508 may transmit data in real-time as it is collected, which may be beneficial for immediate monitoring and analysis.

In some cases, the system may include a Custom Connector function for integrating new devices with different communication protocols. For example, an electrocardiogram (ECG) sensor may be added as a second sensor. The ECG sensor may connect via the same port as the SpO2 sensor 314 (as shown in FIG. 3B), potentially through a branch connector, or through a module that includes connectors for multiple sensors.

The system may be designed to support the addition of new sensing devices. In some cases, new models may be deployed to support additional sensor data provided by these new devices. For instance, if an ECG sensor is added, the mixed linear model 512 may be updated or a new model may be deployed to analyze the ECG data in conjunction with the SpO2 data, potentially providing more comprehensive sleep analysis.

A case study was conducted to evaluate the effectiveness of the proposed system in acquiring SpO2 data efficiently and performing longitudinal analytics using both simulator-generated data and real human subject data. The primary focus was on collecting longitudinal data to estimate the change rate of the Apnea-Hypopnea Index (AHI) over time, which is useful for proactive intervention in managing sleep disorders.

The hardware setup comprised two primary components:
1. Fluke ProSim 8: This device was used to generate SpO2 signals for testing purposes, simulating various health conditions and facilitating data transmission testing from the edge tier to the cloud-based tier.
2. SpO2 device: Equipped with a standard commercial probe, this device measured oxygen levels in the blood and transmitted the collected data to the cloud-based system for further analysis. The SpO2 device incorporated the sensor device 300 described earlier, including the enclosure housing 302, circuit board 306, microcontroller 310, analog front-end 312, and battery 308. The device operated in both batch and streaming modes, allowing for flexible data collection and transmission based on specific monitoring requirements and power considerations.

For the experiments, the system architecture interfaced the AFE4400 with the SpO 2 sensor to acquire photoplethysmograph (PPG) signals indicative of arterial oxygen saturation levels. The SpO 2 sensor analog signal was conditioned by the AFE4400. This integrated analog front-end amplified and filtered the incoming PPG signal, utilizing low-noise operational amplifiers. The AFE4400 employed an onboard analog-to-digital converter to transform the conditioned analog signals into a digitized format suitable for digital signal processing. The resulting digital data stream was relayed to the ESP32 microcontroller via the Serial Peripheral Interface (SPI) bus. The ESP32 functioned as a digital signal processor, applying algorithmic transformations to the digitized PPG data to extrapolate SpO 2 readings, and as the central communication hub, utilizing its integrated wireless communication modules to disseminate the processed SpO 2 readings. Switch buttons were integrated to facilitate user interaction with the ESP32, enabling device activation and the initiation of predefined operational sequences. LEDs provided a visual feedback mechanism, conveying system states such as battery charge levels and data acquisition status. A power management system comprised a power supply unit, battery charging circuitry, and protection mechanism. The circuit layout demonstrated the relative locations of the sensor connector, analog front end, microcontroller, power management, and communication modules. The design emphasized cost-effectiveness for continuous real-time monitoring.

The cloud-based data collection and analytics platform comprised the following components:
1. IoT Gateway: This component managed the communication between the SpO2 device and the cloud-based services, handling protocol conversions and data routing.
2. ThingsBoard: This IoT platform was used for device management, data collection, and initial processing. It served as the central hub for managing the incoming data from the SpO2 device.
3. Apache Spark: Instances of Apache Spark were deployed on virtual machines managed by Spaches Lab at North Dakota State University (NDSU). These instances were used for data processing and analysis, including the implementation of the mixed linear model for AHI prediction.

The software components worked in tandem to facilitate data collection, storage, and analysis. The IoT Gateway received data from the SpO2 device, which was then processed and stored in ThingsBoard. The data was subsequently analyzed using Apache Spark, which implemented the mixed linear model for longitudinal analysis of AHI progression.

An experiment was conducted to collect patient data and train a model. The experiment involved collecting longitudinal data from five cancer patients diagnosed with obstructive sleep apnea (OSA) at Stanford Hospital. The data collection process involved gathering polysomnography (PSG) data, along with various physiological measurements and responses to questionnaires. This included polysomnography (PSG) data, along with various physiological measurements and questionnaires. Table. 1 shows the AHI scores, labeled by medical professionals during patient visits, served as ground truth values for model training and evaluation. Simultaneously, data collected from the device during patient visits were stored in a cloud database at the cloud-based tier. The collected data were used to train a mixed linear model to predict the progression of AHI scores over patient visits. The training process involved feature engineering, model fitting, and optimization. The trained model was evaluated using a separate dataset of longitudinal data from additional cancer patients with OSA. Several performance metrics, including accuracy, precision, recall, and F1-score, were computed to assess the model's effectiveness in predicting AHI scores. Table 1 presents data for five cancer patients, including Patient ID, CancerType/Stage, AHI (Apnea-Hypopnea Index), BMI (Body Mass Index), Sleep Efficiency (%), Mean SpO2(%), and RDI (Respiratory Disturbance Index).

The data in Table 1 shows a range of AHI values across different cancer types and stages. For instance, Patient 1 with Stage IV Prostate Cancer has an AHI of 1.4, while Patient 3 with Stage II Breast Cancer has a substantially higher AHI of 61.1. The table also includes other relevant physiological measurements such as BMI, sleep efficiency, and mean SpO2 levels, which could potentially be used in the analysis of sleep apnea in cancer patients.

The AHI scores in Table 1, labeled by medical professionals during patient visits, served as ground truth values for model training and evaluation. Simultaneously, data collected from the proposed device during patient visits was stored in a cloud database at the cloud-based tier of the system.

TABLE 1

| | | | | Sleep | Mean | |
|---|---|---|---|---|---|---|
| Patient | Cancer | | | Efficiency | SpO2 | |
| ID | Type/Stage | AHI | BMI | (%) | (%) | RDI |
| 1 | Prostate Cancer/IV | 1.4 | 36.62 | 80.7 | 95 | 2.2 |
| 2 | Breast Cancer/II | 0.1 | 23.52 | 93.9 | 98 | 0.4 |
| 3 | Breast Cancer/II | 61.1 | 36.15 | 54.7 | 92.6 | 73.0 |
| 4 | Skin Cancer/I | 15.0 | 35.15 | 71.6 | 93.8 | 22.4 |
| 5 | Breast Cancer/I | 6.6 | 20.84 | 88.3 | 96.0 | 19.5 |

This experiment demonstrates the system's capability to collect, process, and analyze longitudinal sleep data from cancer patients, providing insights into the progression of sleep apnea during cancer treatment.

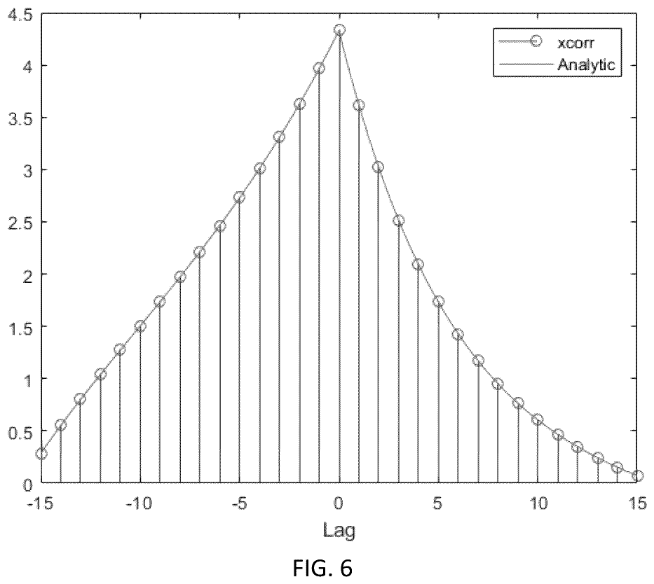

The cross-correlation analysis of SpO2 data collected from the proposed system and a conventional pulse oximeter system reveals a strong correlation, as illustrated in FIG. 6. The graph in FIG. 6 depicts the correlation coefficient on the y-axis ranging from 0 to 4.5 plotted against the time lag on the x-axis ranging from −15 to 15. A blue line with circular markers represents the cross-correlation values labeled as “xcorr” while an orange line labeled as “Analytic” shows the analytical prediction.

The correlation peaks at a value of approximately 4.3 at zero lag, with symmetrical decay on both sides of the peak. This prominent peak at zero lag indicates a high degree of synchronization between the two datasets, suggesting that the proposed system captures SpO2 data with timing accuracy comparable to the conventional system. The symmetrical decay of the correlation on either side of the peak further supports the consistency of the measurements between the two systems.

The strong correlation observed in the cross-correlation analysis implies a high level of data accuracy for the proposed system. The close alignment of the measured data (blue line) with the analytical prediction (orange line) suggests that the proposed system performs in line with theoretical expectations, further validating its accuracy.

These results reflect positively on the reliability of the proposed system. The consistent correlation across different time lags indicates that the system maintains accuracy over extended periods, which is particularly important for longitudinal monitoring of cancer patients during treatment.

To further evaluate the system's performance, Table 2 presents a detailed comparison of data loss and latency measurements for SpO2 transmission in different health conditions:

TABLE 2

| | Data Loss (%) | Latency (ms) |
|---|---|---|
| Health Conditions | x̅ ± s | x̅ ± s |
| Normal | 0.389 ± 0.229 | 760 ± 421 |
| Hypertension | 0.135 ± 0.320 | 463 ± 322 |
| Hypotension | 0.1231 ± 0.3061 | 631 ± 386 |

The data presented in Table 2 provides insights into the system's performance under various health conditions. For normal health conditions, the system exhibits a mean data loss of 0.389% with a standard deviation of 0.229%, and a mean latency of 760 ms with a standard deviation of 421 ms. In hypertensive conditions, the system demonstrates lower data loss (0.135%±0.320%) and reduced latency (463 ms±322 ms). For hypotensive conditions, the system maintains low data loss (0.1231%±0.3061%) with moderate latency (631 ms±386 ms).

These results may demonstrate the robustness of the proposed system across different health conditions. The consistently low data loss percentages across all conditions indicate reliable data transmission, which supports continuous monitoring of cancer patients. The varying latency values across different health conditions suggest that the system adapts to different physiological states, potentially optimizing its performance based on the patient's condition.

The low data loss and reasonable latency values observed in the proposed system correspond well with the performance expectations of gold-standard testing modalities. In some cases, the system's ability to maintain data integrity and timely transmission across various health conditions is comparable to or even exceeds the capabilities of conventional pulse oximetry systems.

Table 3 displays the predicted AHI rates for each patient visit using the mixed linear model, alongside the corresponding historical AHI rates obtained from the PSG system. The comparison between the predicted and historical AHI rates demonstrates the effectiveness of the longitudinal analysis for predicting patient AHI condition.

TABLE 3

| | | Historical | | | | F1- |
|---|---|---|---|---|---|---|
| | Predicted | AHI | Accuracy | Precision | Recall | Score |
| Visit | AHI | (PSG) | (%) | (%) | (%) | (%) |
| 1 | 12.3 | 11.8 | 89.2 | 88.1 | 92.3 | 90.01 |
| 2 | 14.5 | 13.9 | 91.7 | 89.8 | 94.2 | 91.9 |
| 3 | 16.8 | 17.2 | 88.5 | 87.2 | 91.8 | 89.5 |
| 4 | 18.2 | 20.5 | 90.1 | 88.9 | 92.7 | 90.8 |
| 5 | 20.1 | 21.7 | 87.9 | 86.7 | 90.5 | 88.5 |

The longitudinal analysis results reveal strong correlations between the predicted AHI rates and the historical AHI rates, potentially indicating the reliability of the machine learning model. Evaluation metrics such as accuracy, precision, recall, and F1-score are computed to assess the model's performance, with promising results observed across all metrics.

In some implementations, the implications of the longitudinal analysis results extend to clinical practice, where proactive intervention strategies can be devised based on the predicted AHI rates to manage and mitigate the progression of OSA in cancer patients undergoing treatment. This highlights the significance of leveraging longitudinal data and advanced analytics techniques in improving patient care outcomes.

The mixed linear model trained on longitudinal cancer patient data collected from the device provides a comprehensive view of dynamic changes in physiological parameters over time. The close match between the predicted and historical AHI rates demonstrate the effectiveness of the longitudinal analysis for predicting patient AHI condition.

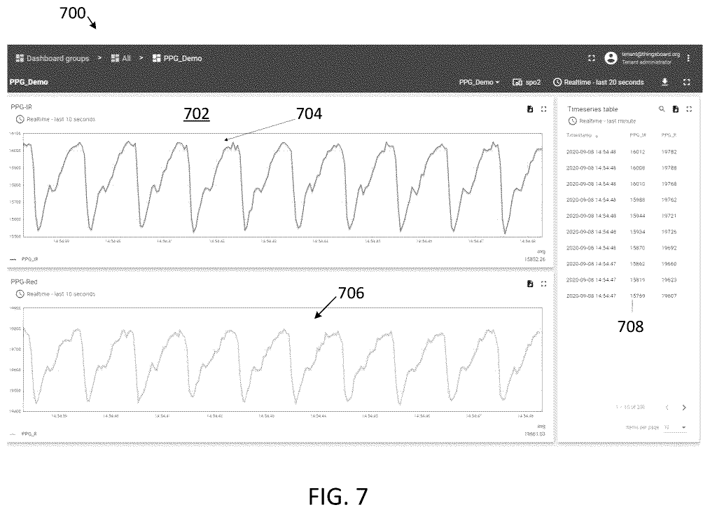

The monitoring dashboard 700 illustrated in FIG. 7 provides an example of how the data collected and processed by the system described in the previous figures can be presented to healthcare professionals. This dashboard displays real-time physiological signal data through graphs and data tables, offering a visual representation of the information gathered by the sensor device 300 (shown in FIG. 3) and processed using the method 400 (outlined in FIG. 4).

The IR dashboard 702 displays an infrared PPG signal graph, which corresponds to the SpO2 signals collected at block 102 in FIG. 1. Adjacent to the IR dashboard 702, a PPG-IR 704 presents the processed PPG-IR waveform data over time, reflecting the data processing steps described in blocks 106 and 110 of FIG. 1. Below these graphs, a red light dashboard 706 presents the red light PPG signal measurements. The red light measurements, in conjunction with the infrared measurements, are used to calculate oxygen saturation levels in the blood, as may occur in block 424 of FIG. 4.

A data table 708 is positioned on the right side of the monitoring dashboard 700. This table displays numerical values corresponding to the graphical data, providing measurements for parameters such as heart rate and SpO2 levels. These values may be derived from the data processing and analysis steps outlined in FIG. 4 and FIG. 5.

In some implementations, the monitoring dashboard 700 serves as a visual representation of the data flow and analysis processes depicted in the sequence diagram 500 of FIG. 5. It provides healthcare professionals with a user-friendly interface to access and interpret the results of the data analysis 528 performed by the mixed linear model 512.

The monitoring dashboard 700 provides healthcare professionals with a view of physiological data collected from cancer patients undergoing treatment. FIG. 7 illustrates an example of a monitoring dashboard 700 that displays real-time physiological signal data through graphs and data tables.

The monitoring dashboard 700 may provide a longitudinal progression of the apnea measure through a dedicated section displaying Apnea-Hypopnea Index (AHI) differences over time. This section may feature a line graph showing the patient's AHI values plotted against dates, allowing healthcare professionals to track changes in sleep apnea severity over weeks or months. The graph may use color-coding to indicate different severity levels of sleep apnea, potentially using green for normal ranges, yellow for mild apnea, orange for moderate apnea, and red for severe apnea.

As a user service, the dashboard may offer interactive features to enhance data analysis capabilities. Healthcare professionals may be able to use date selection tools to view data for specific nights or sleep sessions. For example, a calendar widget may allow doctors to choose a particular date, which may then populate all graphs and tables with information from that specific sleep session.

In addition to the dashboard, other user services may be implemented to enhance the utility of the collected data. One such service may involve storing the longitudinal data in a secure, cloud-based repository. This storage solution may allow for easy retrieval of historical data and may facilitate data sharing between authorized healthcare providers. The storage service may also implement automatic backup and encryption protocols to ensure data integrity and patient privacy.

Another user service may focus on incorporating the collected sleep apnea data into a patient's electronic medical record (EMR). This integration may allow for a more comprehensive view of the patient's health status, potentially improving care coordination among different specialists. The EMR integration service may include features such as automatic data synchronization and customizable alerts for significant changes in a patient's sleep apnea status.

The system may also offer a user service for presenting the longitudinal data in alternative formats, such as through detailed reports or summary statistics. These reports may include trend analyses, comparing the patient's sleep apnea progression to population norms or expected trajectories based on their cancer type and treatment. The reporting service may offer customizable templates, allowing healthcare providers to tailor the information presentation to their specific needs or preferences.

The monitoring dashboard 700 includes an IR dashboard 702 that displays an infrared PPG signal graph. This graph shows the intensity of infrared light absorption over time, which correlates with blood volume changes in the tissue being monitored. Adjacent to the IR dashboard 702, a PPG-IR 704 presents the processed PPG-IR waveform data over time. This processed waveform provides a representation of the patient's pulse and blood flow characteristics. Below these graphs, a red light dashboard 706 presents the red light PPG signal measurements. The red light measurements, in conjunction with the infrared measurements, are used to calculate oxygen saturation levels in the blood. By displaying both the infrared and red light signals, the monitoring dashboard 700 allows healthcare professionals to visually assess the quality and consistency of the raw data used for SpO2 calculations.

A data table 708 is positioned on the right side of the monitoring dashboard 700. The data table 708 displays numerical values corresponding to the graphical data, providing measurements for parameters such as heart rate, SpO2 levels, and potentially derived metrics like respiratory rate.

In some cases, the monitoring dashboard 700 includes a section dedicated to displaying Apnea-Hypopnea Index (AHI) differences over time. This section may feature a line graph showing the patient's AHI values plotted against dates, allowing healthcare professionals to track changes in sleep apnea severity over weeks or months. The graph may use color-coding to indicate different severity levels of sleep apnea, potentially using green for normal ranges, yellow for mild apnea, orange for moderate apnea, and red for severe apnea.

The monitoring dashboard 700 also incorporates real-time data presentation capabilities. A dedicated area of the dashboard displays continuously updating values for heart rate, blood oxygen levels, and respiratory rate. These values may refresh at regular intervals, such as every 30 seconds, to provide insights into the patient's current physiological state.

To enhance usability and data analysis capabilities, the monitoring dashboard 700 includes interactive features. Healthcare professionals may be able to use date selection tools to view data for specific nights or sleep sessions. For example, a calendar widget may allow doctors to choose a particular date, which may then populate all graphs and tables with information from that specific sleep session.

The monitoring dashboard 700 also provides tools for selecting custom time periods for detailed analysis. Slider controls or input fields may allow users to define start and end times for a period of interest. Upon selection, the dashboard may automatically update all displayed data to reflect the chosen time range, enabling focused examination of specific events or trends.

In some implementations, the monitoring dashboard 700 supports data comparison features. Healthcare professionals may be able to overlay data from multiple nights or compare data from different time periods side by side. This functionality may facilitate the identification of patterns or changes in the patient's sleep quality and physiological parameters over the course of their cancer treatment.

The monitoring dashboard 700 may also include alert indicators for abnormal readings or concerning trends. These alerts may be visually prominent, potentially using color-coded icons or flashing elements to draw attention to parameters that fall outside of predefined normal ranges. This feature may help healthcare professionals quickly identify potential issues that may require immediate attention or further investigation.

By providing a comprehensive and interactive display of both real-time and historical patient data, the monitoring dashboard 700 may serve as a valuable tool for healthcare professionals monitoring cancer patients for sleep-related issues during treatment. The combination of visual graphs, numerical data, and interactive features may support efficient data analysis and informed decision-making in patient care.

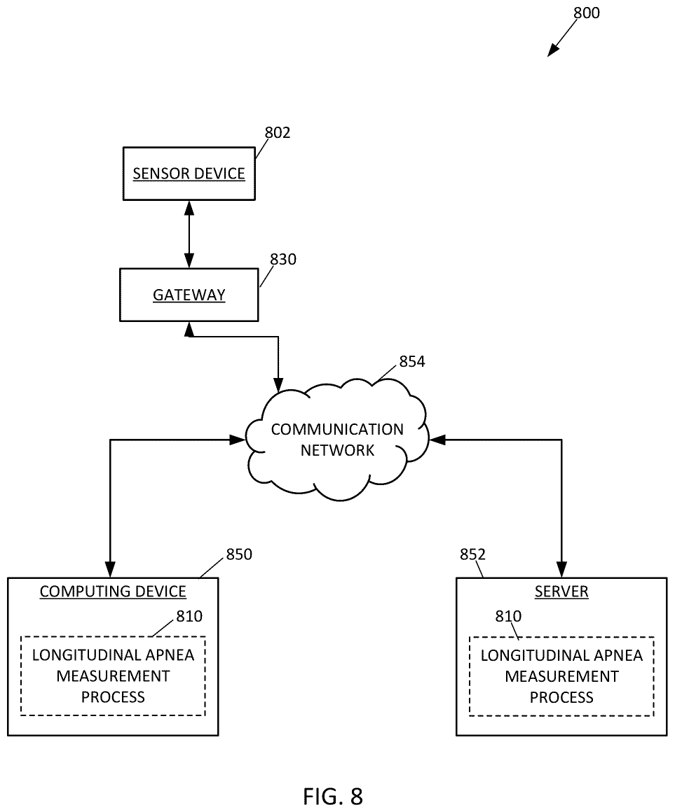

FIG. 8 illustrates an example of a sleep monitoring system 800 in accordance with some embodiments described in the present disclosure. As shown in FIG. 8, a sensor device 802 may collect sleep-related data from a cancer patient undergoing treatment. The sensor device 802 may communicate this data to a gateway device 830, which may then transmit the data to a computing device 850 and/or a server 852 over a communication network 854. In some cases, the computing device 850 and/or server 852 may execute at least a portion of an apnea measurement process 810 to process the data received from the sensor device 802 and perform sleep apnea analysis.

The sleep monitoring system 800 may implement various components and processes described in previous figures. For example, the sensor device 802 may incorporate elements of the sensor device 300 shown in FIG. 3, such as the SpO2 sensor 314 and microcontroller 310. The gateway device 830 may perform functions similar to those described for the gateway device 230 in FIG. 2, including processing and formatting SpO2 data.

In some cases, the computing device 850 may be any suitable computing device or combination of devices, such as a desktop computer, a laptop computer, a tablet computer, or a virtual machine being executed by a physical computing device. The server 852 may be a dedicated server computer or a cloud-based computing resource. Both the computing device 850 and server 852 may implement components of the cloud-based tier described in FIG. 2, such as the data manager 246 and analysis service 274.

The sensor device 802 may be designed to collect various types of sleep-related data, potentially including SpO2 signals, heart rate, respiratory rate, and body movement. This data collection may align with the processes outlined in the method 100 of FIG. 1, particularly the data collection described in block 102. The sensor device 802 may be positioned in the patient's sleeping environment and may communicate data to the gateway device 830, which may then relay the data to the computing device 850 and/or server 852 via the communication network 854.

The communication network 854 may be any suitable communication network or combination of communication networks. For example, the communication network 854 may include a Wi-Fi network, a cellular network (e.g., a 4G or 5G network), or other types of wireless or wired networks. In some cases, the communication network 854 may be a local area network, a wide area network, or a combination of different network types. The communication links between the devices in FIG. 8 may each be any suitable communications link or combination of communications links, such as wired links, fiber optic links, Wi-Fi links, Bluetooth links, or cellular links.

The apnea measurement process 810 executed by the computing device 850 and/or server 852 may incorporate various analytical steps described in previous figures. For instance, the process may include feature extraction from SpO2 data and application of a mixed linear model, as outlined in blocks 110 and 112 of FIG. 1. The results of this analysis may be used to provide a longitudinal progression of apnea measures for the cancer patient over multiple sleep sessions.

In some implementations, the computing device 850 may host a user interface similar to the monitoring dashboard 700 shown in FIG. 7. This interface may allow healthcare professionals to view real-time and historical sleep data, including SpO2 levels, heart rate, and derived apnea measures. The server 852 may handle data storage and more intensive computational tasks, potentially utilizing components like the storage system 244 and analysis service 274 described in FIG. 2.

By integrating these various components and processes, the sleep monitoring system 800 may provide a comprehensive solution for monitoring and analyzing sleep patterns in cancer patients undergoing treatment. The system may enable continuous, long-term data collection and analysis, potentially improving the detection and management of sleep-related issues in this patient population.

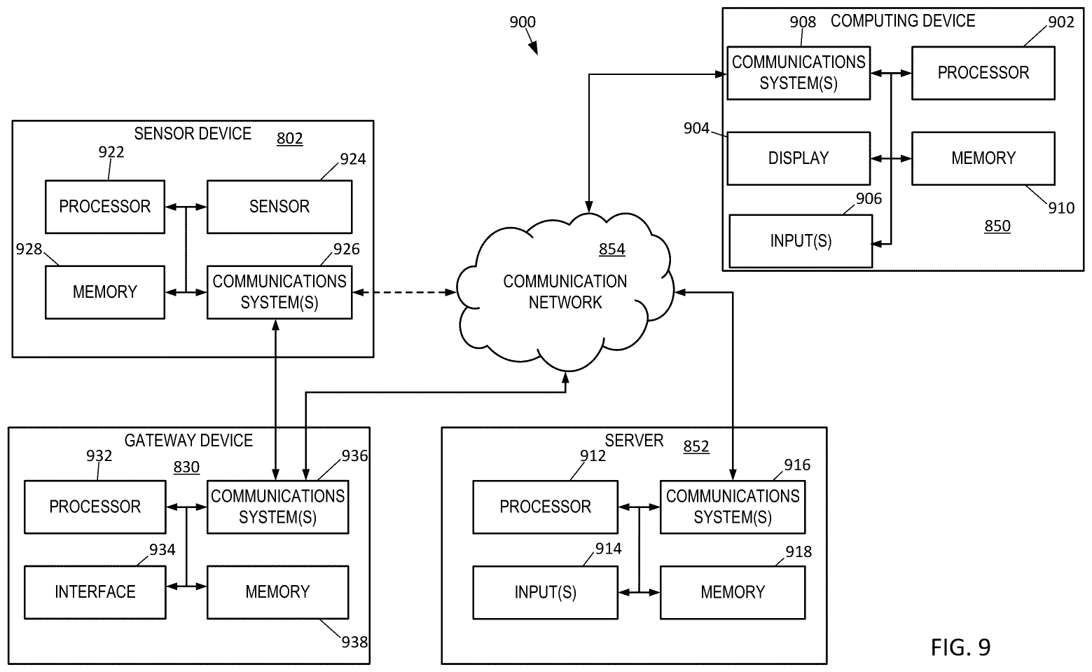

Referring now to FIG. 9, an example of a system architecture 900 that can be used to implement the sensor device 802, gateway device 830, computing device 850, and server 852 in accordance with some embodiments of the systems and methods described in the present disclosure is shown. As shown in FIG. 9, in some embodiments, the computing device 850 may include a processor 902, a display 904, one or more input/output devices 906, a communications system 908, and/or memory 910. In some embodiments, the processor 902 may be any suitable hardware processor or combination of processors, such as a central processing unit (CPU), a graphics processing unit (GPU), and so on. In some embodiments, the display 904 may include any suitable display devices, such as a liquid crystal display (LCD) screen, a light-emitting diode (LED) display, an organic LED (OLED) display, a computer monitor, a touchscreen, and so on. In some embodiments, the input/output devices 906 may include any suitable input devices and/or sensors that can be used to receive user input, such as a keyboard, a mouse, a touchscreen, a microphone, and so on.

In some embodiments, the communications system 908 may include any suitable hardware, firmware, and/or software for communicating information over the communication network 854 and/or any other suitable communication networks. For example, the communications system 908 may include one or more transceivers, one or more communication chips and/or chip sets, and so on. In a more particular example, the communications system 908 may include hardware, firmware, and/or software that can be used to establish a Wi-Fi connection, a Bluetooth connection, a cellular connection, an Ethernet connection, and so on.

In some embodiments, the memory 910 may include any suitable storage device or devices that can be used to store instructions, values, data, or the like, that can be used, for example, by the processor 902 to present content using the display 904, to communicate with the server 852 via the communications system 908, and so on. The memory 910 may include any suitable volatile memory, non-volatile memory, storage, or any suitable combination thereof. For example, the memory 910 may include random-access memory (RAM), read-only memory (ROM), electrically programmable ROM (EPROM), electrically erasable ROM (EEPROM), other forms of volatile memory, other forms of non-volatile memory, one or more forms of semi-volatile memory, one or more flash drives, one or more hard disks, one or more solid state drives, one or more optical drives, and so on.

In some embodiments, the server 852 may include a server processor 912, a server input interface 914, a server communications system 916, and/or server memory 918. The server processor 912 may be similar to the processor 902 of the computing device 850, capable of executing instructions stored in the server memory 918. The server input interface 914 may allow for the input of data or commands to the server 852. The server communications system 916 may be similar to the communications system 908 of the computing device 850, enabling communication with other devices in the system architecture 900.

The sensor device 802 may include a sensor processor 922, a sensor 924, a sensor communications system 926, and/or sensor memory 928. The sensor processor 922 may control the operations of the sensor device 802, including data collection and transmission. The sensor 924 may be a SpO2 sensor or other physiological sensor capable of collecting data from a patient. The sensor communications system 926 may enable the sensor device 802 to transmit collected data to the gateway device 830 or other components of the system.

The gateway device 830 may include a gateway processor 932, a gateway interface 934, a gateway communications system 936, and/or gateway memory 938. The gateway processor 932 may manage the operations of the gateway device 830, including data processing and routing. The gateway interface 934 may allow for configuration and control of the gateway device 830. The gateway communications system 936 may enable communication between the gateway device 830 and other components of the system architecture 900.

In some embodiments, the system architecture 900 may be configured to perform the apnea monitoring service described in previous figures. For example, the server 852 or the computing device 850 may execute instructions stored in their respective memories to perform feature extraction on formatted SpO2 data received from the sensor device 802 via the gateway device 830. This feature extraction may include extracting at least one of a sleep architecture feature, a sleep arousal feature, an oxygen saturation feature, or a heart rate feature from the formatted SpO2 data.

The extracted features may then be used as inputs to a mixed-linear model, which may be applied to determine an apnea measure. In some cases, the mixed-linear model may be implemented as a set of instructions stored in the server memory 918 or the memory 910 of the computing device 850, and executed by the respective processors. Following the application of the mixed-linear model, a logarithmic transformation may be applied to the output to determine the final apnea measure. This transformation may help normalize the data and improve the model's performance across different patients and conditions.

The components of the system architecture 900 may work together to collect, transmit, process, and analyze physiological data for sleep apnea monitoring in cancer patients. For example, the sensor device 802 may collect SpO2 data from a patient and transmit this data to the gateway device 830. The gateway device 830 may then process and format this data before sending it to the server 852 or computing device 850 for analysis. The server 852 or computing device 850 may then perform the feature extraction, apply the mixed-linear model, and perform the logarithmic transformation to determine the apnea measure.

In some embodiments, the memory 910, server memory 918, sensor memory 928, and gateway memory 938 may have encoded thereon, or otherwise stored therein, computer programs for controlling operation of their respective devices. In such embodiments, the respective processors may execute at least a portion of the computer programs to perform the methods described herein, such as the method 100 illustrated in FIG. 1 or the method 400 illustrated in FIG. 4.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” “engine,” “manager,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on). For instance, as shown in FIG. 2, the edge device 202 includes various components such as sensors 208, protocol interfaces, gateway interface 238, software processes including model 288 and analysis service 274. These components work together to collect and transmit SpO2 data, demonstrating how multiple components can be integrated within a single system to perform complex functions.

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

A number of implementations have been described. Nevertheless, it will be understood that various modifications may be made without departing from the spirit and scope of the disclosure. Accordingly, other implementations are within the scope of the following claims.

1. A system, comprising:
a device to collect SpO2 signals from a patient undergoing treatment over a plurality of sleep sessions;
a gateway to connect to the device via a local connection to receive the SpO2 signals, process the SpO2 signals to generate formatted SpO2 data, and to transmit the formatted SpO2 data;
an apnea monitoring service to determine an apnea measure for the patient based on the formatted SpO2 data; and
a user service to provide a longitudinal progression of the apnea measure over the plurality of sleep sessions.

2. The system of claim 1, wherein the device comprises a SpO2 sensor and a microcontroller to process raw SpO2 signals from the SpO2 sensor and transmit the processed SpO2 signals to the gateway via a wireless communication protocol.

3. The system of claim 2, wherein the gateway is to control the device to enter a batch mode or a stream mode, wherein in the batch mode the device accumulates SpO2 signals over a predetermined time period before transmitting to the gateway, and in the stream mode the device transmits SpO2 signals to the gateway in real-time as they are collected.

4. The system of claim 1, wherein the apnea monitoring service performs feature extraction on the formatted SpO2 data to determine the apnea measure.

5. The system of claim 4, wherein the feature extraction includes extracting at least one of a sleep architecture feature, a sleep arousal feature, an oxygen saturation feature, or a heart rate feature from the formatted SpO2 data.

6. The system of claim 5, wherein the apnea monitoring service applies a mixed-linear model to the extracted features to determine the apnea measure.

7. The system of claim 6, wherein the apnea monitoring service applies a logarithmic transformation to the output of the mixed-linear model to determine the apnea measure.

8. A method, comprising:
collecting, over a plurality of sleep sessions, SpO2 signals using an SPO2 sensing device from a patient undergoing treatment;
receiving the SpO2 signals at a gateway via a local connection;
processing the SpO2 signals at the gateway to generate formatted SpO2 data;
transmitting the formatted SpO2 data from the gateway;
determining an apnea measure for the patient based on the formatted SpO2 data; and
providing a longitudinal progression of the apnea measure over the plurality of sleep sessions.

9. The method of claim 8, further comprising:
processing raw SpO2 signals from a SpO2 sensor using a microcontroller; and
transmitting the processed SpO2 signals to the gateway via a wireless communication protocol.

10. The method of claim 9, further comprising:
controlling the SPO2 sensing device to enter a batch mode or a stream mode, wherein:
in the batch mode, accumulating SpO2 signals over a predetermined time period before transmitting to the gateway; and
in the stream mode, transmitting SpO2 signals to the gateway in real-time as they are collected.

11. The method of claim 8, wherein determining the apnea measure comprises performing feature extraction on the formatted SpO2 data.

12. The method of claim 11, wherein the feature extraction includes extracting at least one of a sleep architecture feature, a sleep arousal feature, an oxygen saturation feature, or a heart rate feature from the formatted SpO2 data.

13. The method of claim 12, further comprising applying a mixed-linear model to the extracted features to determine the apnea measure.

14. The method of claim 13, further comprising applying a logarithmic transformation to the output of the mixed-linear model to determine the apnea measure.

15. A non-transitory computer-readable medium storing instructions that, when executed by one or more processors, cause the one or more processors to perform operations comprising:
collecting SpO2 signals using an SPO2 sensing device from a patient undergoing treatment over a plurality of sleep sessions;
receiving the SpO2 signals at a gateway via a local connection;
processing the SpO2 signals at the gateway to generate formatted SpO2 data;
transmitting the formatted SpO2 data from the gateway;
determining an apnea measure for the patient based on the formatted SpO2 data; and
providing a longitudinal progression of the apnea measure over the plurality of sleep sessions.

16. The non-transitory computer-readable medium of claim 15, wherein the operations further comprise:
processing raw SpO2 signals from a SpO2 sensor using a microcontroller; and
transmitting the processed SpO2 signals to the gateway via a wireless communication protocol.

17. The non-transitory computer-readable medium of claim 16, wherein the operations further comprise:
controlling the SPO2 sensing device to enter a batch mode or a stream mode, wherein:
in the batch mode, accumulating SpO2 signals over a predetermined time period before transmitting to the gateway; and
in the stream mode, transmitting SpO2 signals to the gateway in real-time as they are collected.

18. The non-transitory computer-readable medium of claim 15, wherein determining the apnea measure comprises performing feature extraction on the formatted SpO2 data.

19. The non-transitory computer-readable medium of claim 18, wherein the feature extraction includes extracting at least one of a sleep architecture feature, a sleep arousal feature, an oxygen saturation feature, or a heart rate feature from the formatted SpO2 data.

20. The non-transitory computer-readable medium of claim 19, wherein the operations further comprise:
applying a mixed-linear model to the extracted features to determine the apnea measure; and
applying a logarithmic transformation to the output of the mixed-linear model to determine the apnea measure.
